(19) United States
(12) Patent Application Publication
(10) Pub. No.: US 2026/0061173 A1
(43) Pub. Date: Mar. 5, 2026

(54) ARGININE MODIFIED CHITOSAN/POLYVINYL ALCOHOL HYDROGEL MICRONEEDLE TRANSDERMAL DELIVERY PATCHES FOR WOUND HEALING

(71) Applicants: Muhammad Raza Shah, Karachi (PK); Muhammad Hasnain, Karachi (PK); Maria Khalid, Karachi (PK)

(72) Inventors: Muhammad Raza Shah, Karachi (PK); Muhammad Hasnain, Karachi (PK); Maria Khalid, Karachi (PK)

(21) Appl. No.: 18/818,588

(22) Filed: Aug. 29, 2024

(51) Int. Cl. A61M 37/00; A61K 9/00; A61K 31/12; A61K 47/08; A61K 47/32; A61K 47/36

(52) U.S. Cl. CPC A61M 37/0015; A61K 9/0014; A61K 31/12; A61K 47/08; A61K 47/32; A61K 47/36; A61M 2037/0023

(57) ABSTRACT

The present invention relates to a microneedle-based transdermal delivery patch fabricated from a hydrogel composed of polyvinyl alcohol (PVA), arginine-modified chitosan, and a crosslinker. The arginine-modified chitosan is prepared by reacting a chitosan solution with an arginine solution preactivated with N-hydroxy succinimide and 1-Ethyl-3-(3-dimethylaminopropyl)carbodiimide at a specific pH. The hydrogel's formulation further comprises a PVA concentration ranging from 10% to 12% (w/v). arginine-modified chitosan and PVA solution are mixed in a 1:2 ratio by volume and are crosslinked using trimethyl orthoformate (TMOF). The patch is further loaded with curcumin, with a concentration of 120μg per patch, demonstrating a 70% release over 48 hours in a controlled manner. This invention represents the first application of PVA crosslinked arginine-modified chitosan hydrogel loaded with curcumin specifically for animal wound healing, providing enhanced wound healing and antibacterial efficacy.

### BACKGROUND OF THE INVENTION

### 1. Field of Invention

The present invention pertains to a hydrogel microneedle based transdermal delivery drug delivery patch. This patch is fabricated from a hydrogel comprising polyvinyl alcohol (PVA), arginine-modified chitosan, and a crosslinker. The invention also encompasses the composition for manufacturing such a patch. Specifically, it focuses on utilizing the patch, loaded with curcumin, for effective wound healing. This invention is relevant to the fields of transdermal drug delivery systems as well as dermatological and wound care products.

### 2. Description of Related Art

The use of microneedles for transdermal drug delivery has gained significant attention in recent years due to their minimally invasive nature as well as their potential to deliver therapeutic agents directly into the skin. Microneedle patches have been extensively studied for various applications including drug delivery. More recently, their potential in wound healing has been explored, leveraging their ability to deliver active ingredients locally to the wound site in a controlled manner.

Chitosan, a biopolymer derived from chitin, has been widely recognized for its inherent antimicrobial properties, biocompatibility, biodegradability making it a promising candidate for wound healing applications. Due to their moisture-retaining capabilities and their ability to promote wound healing, chitosan-based hydrogels have been extensively explored as wound dressings. Previous patents, such as U.S. Pat. No. 8,158,845B2, EP3562520B1, CN219021927U, have disclosed chitosan-based formulations for wound healing, emphasizing their effectiveness in maintaining a moist wound environment and promoting tissue regeneration. The modification of chitosan with arginine, an amino acid known for its role in wound healing and immune response, has been proposed to enhance the bioactivity and antibacterial activity of chitosan. Arginine-modified chitosan hydrogels have the potential to improve cell proliferation, collagen synthesis, and overall wound healing outcomes.

Polyvinyl alcohol (PVA) is another polymer commonly used in hydrogel formulations due to its excellent film-forming properties, biocompatibility, and mechanical strength. PVA-based hydrogels have been employed in wound care for their ability to provide a protective barrier while allowing for the controlled release of therapeutic agents. The combination of PVA with chitosan in hydrogel formulations has been explored in several studies and patents, including CN110760077B, which discusses PVA and chitosan based hydrogels for wound healing applications.

Curcumin, a natural polyphenolic compound derived from the turmeric plant, has been extensively studied for its anti-inflammatory, antioxidant, and antimicrobial properties. These attributes make curcumin a valuable therapeutic agent for wound healing. However, curcumin's poor solubility and stability in physiological conditions have limited its direct application. Previous inventions, such as Spanish patent ES2885052T3, have explored curcumin for therapeutic applications, including wound healing.

Microneedles, particularly those fabricated from biocompatible polymers, have emerged as an effective platform for transdermal and intradermal drug delivery. Their application in wound healing has been explored in various contexts, where they offer the advantage of painless delivery, reduced risk of infection, and enhanced therapeutic efficacy through localized treatment. Patents such as US Patent Application No. US20050143713A1 have discussed the use of microneedle patches for the delivery of therapeutic agents. While arginine-modified chitosan has been explored in some contexts, there has been limited disclosure in the patent and non patent literature regarding its use alone and in combination with other polymers in microneedle-based delivery systems for wound healing.

The precise subject of the present invention are microneedle patches made from an arginine modified chitosan/PVA hydrogel, one embodiment of which has been loaded with the antibacterial, antioxidant and anti-inflammatory ingredient curcumin. While various components of this invention have been explored in isolation—such as chitosan/PVA hydrogels, curcumin delivery systems, and microneedles—this novel combination, particularly the fabrication of microneedles with the combination of PVA with arginine-modified chitosan, represents a significant advancement over existing technologies. The present invention represents an innovative approach by combining arginine-modified chitosan with PVA as a hydrogel matrix, and formed into microneedle patches which may additionally incorporate therapeutics to specifically utilize for wound healing applications. One embodiment was loaded with curcumin. This invention addresses several challenges associated with wound healing, including the need for localized, controlled delivery of bioactive compounds, and the enhancement of wound healing processes through the synergistic effects of chitosan arginine modification and curcumin's therapeutic properties. The integration of these components into a microneedle patch offers a unique and effective solution for promoting wound healing, distinguishing it from prior art and providing a strong foundation for this patent application.

### SUMMARY OF THE INVENTION

This invention relates to the development of hydrogel microneedle transdermal delivery patches fabricated from a hydrogel comprising cross-linked polyvinyl alcohol and arginine-modified chitosan. In one embodiment of the invention, these patches are further loaded with curcumin, emphasizing their role in innovative transdermal drug delivery systems.

Chitosan (CH) (5 mL of 0.3% w/v) was prepared by dissolving CH in 1% acetic acid (v/v) to form a homogeneous solution at 25° C. Arginine (ARG) aqueous solution (0.04 mmol, 3 mL) was supplemented with N-hydroxysuccinimde (NHS) (0.03 mmol) and 1-Ethyl-3-(3-dimethylaminopropyl) carbodiimide (EDC) (0.09 mmol) and allowed to stir for 1 hour to activate the carboxyl group of ARG. After 1 hour activated ARG solution was added to the CH solution. The pH of the CH solution was then adjusted to 5.5 using HCl or NaOH or base if necessary. The resulting mixture was then stirred for 24 h. After stirring, the mixture was transferred to a dialysis membrane and dialyzed against distilled water for 72 hours with frequent changes of distilled water to remove unreacted materials and byproducts, yielding chitosan-arginine solution (CH-ARG).

Polyvinyl alcohol (PVA) (10-12% w/v) was made by dissolving the requisite amount distilled water, and the solution was heated to homogeneity at 60° C. with continuous stirring. The chitosan-arginine solution (CH-ARG) as prepared above and PVA solution were mixed in 1:2 ratio. Trimethylorthoformate (TMOF) 4% v/v was added with respect to the volume of the total polymeric mixture. The resulting hydrogel was referred to as CH-ARG/PVA hydrogel.

Microneedle (MN) patches were fabricated by the one-layer centrifugation method. 5 mL CH-ARG/PVA hydrogel into polydimethylsiloxane (PDMS) molds (15×15 pyramids, each with a height of 345 μm and a base length of 250 μm). The molds were centrifuged at 1000 rpm for 10 min, then dried at 25° C for 24 hour. After that, the resulting CH-ARG/PVA MN patches, hereafter referred to as blanks-MNs were carefully removed.

The CH-ARG/PVA/CUR hydrogel was produced by simply adding a methanolic Curcumin (CUR) solution (1 mL of 2 mg/mL solution) to 5 mL of CH-ARG/PVA hydrogel while stirring. The CUR based MN patches were fabricated similarly by one-layer centrifugation method. CH-ARG/PVA/CUR hydrogel (5 mL) was poured into polydimethylsiloxane (PDMS) molds (15×15 pyramids, each with a height of 345 μm and a base length of 250 μm). The molds were centrifuged at 1000 rpm for 10 min and dried at 25° C. for 24 hours. After that, the resulting patches, each containing 120 μg of curcumin were carefully removed.

The microneedle patches were found to have an antioxidant potential and inhibiting bacterial growth in an in vitro analysis against S. aureus and E. Coli, the bacterial strains primarily involved in wound infection.

It was also observed that the CUR-loaded MNs greatly improved wound healing potential via tissue regeneration and collagen deposition.

### BRIEF DESCRIPTION OF THE DRAWINGS

FIG. 1: ¹H NMR (A) and FTIR (B) spectra of ARG, CH, and CH-ARG.

FIG. 2: FTIR (A) and Swelling analysis (B) of CH-ARG/PVA and CH-ARG/PVA/CUR hydrogels.

FIG. 3: SEM images of the blank-MNs after applying pressures of 100 g (A), 200g (B), 500 g (C), and 1000 g (D). SEM images of the CUR-MNs after applying pressures of 100 g (E), 200 g (F), 500 g (G), and 1000 g (H).

FIG. 4 Optical microscopic (A) and SEM images of MNs (B, C, and D).

FIG. 5: In vitro drug release profile of CUR-MNs (A) and in vitro antioxidant activity of blank-MNs and CUR-MNs (B) at different time interval.

FIG. 6: Zone of inhibition of blank-MNs against E. coli (A), S. aureus (B), and CUR-MNs against E. coli (C), and S. aureus (D).

FIG. 7: % Cell viability of control and MNs against primary mouse fibroblasts cells after 24 h.

FIG. 8: Macroscopic visualization images of wound for blank-MNs, CUR-MNs, and control groups at 0, 3, 6, 9 and 14 days.

FIG. 9: Wound reduction percentage at different time interval (**P<0.01; ***P<0.001).

FIG. 10: Histological analysis of the wound tissue at days 3, 6, 9 and 14 post wound treatment under 40× magnification after being stained with Hematoxylin-Eosin (A) and Masson's trichrome (B).

### DETAILED DESCRIPTION OF THE INVENTION

This invention pertains to the development of microneedle (MN) patches fabricated from a hydrogel composed of cross-linked polyvinyl alcohol (PVA) and arginine-modified chitosan (ARG-CH).

To prepare the hydrogel, Chitosan (CH) (5 mL of 0.3% (w/v)) was prepared by dissolving CH in 1% acetic acid (v/v) to form a homogeneous solution at 25° C. An arginine (ARG) aqueous solution (0.04 mmol, 3 mL) was supplemented with N-hydroxysuccinimide (NHS) (0.03 mmol) and 1-ethyl-3-(3-dimethylaminopropyl) carbodiimide (EDC) (0.09 mmol) and allowed to stir for 1 hour to activate the carboxyl group of ARG. After 1 hour, the activated arginine solution was added to the CH solution. The pH of the chitosan solution was then adjusted to 5.5 using hydrochloric acid (HCl) or sodium hydroxide (NaOH), as needed. The resulting mixture was stirred for 24 hours and then transferred to a dialysis membrane. It was dialyzed against distilled water for 72 hours with frequent changes of distilled water to remove unreacted materials and byproducts yielding chitosan-arginine solution (CH-ARG).

Polyvinyl alcohol (PVA) solution (10-12% w/v) was prepared by dissolving the requisite amount in distilled water, and the solution was heated to 60° C. with continuous stirring to achieve homogeneity. The chitosan-arginine (CH-ARG) solution, as prepared above, and the PVA solution were mixed in a 1:2 ratio by volume. Trimethyl orthoformate (TMOF) at 4% (v/v) was added relative to the total mixture volume resulting in the formation of CH-ARG/PVA hydrogel.

Microneedle (MN) patches were fabricated using a one-layer centrifugation method. Specifically, 5 mL of CH-ARG/PVA hydrogel was poured into polydimethylsiloxane (PDMS) molds containing an array of 15×15 pyramids, each with a height of 345 μm and a base length of 250 μm. The molds were centrifuged at 1000 rpm for 10 minutes, then dried at 25° C. for 24 hours. After drying, the resulting patches, hereafter referred to as blank-MNs in the specification and drawing, were carefully removed.

One embodiment of the invention involved further loading of curcumin (CUR) for advanced transdermal drug delivery system. For the purpose, the CH-ARG/PVA/CUR hydrogel was produced by adding a methanolic curcumin (CUR) solution (1 mL of a 2 mg/mL solution) to 5 mL of CH-ARG/PVA hydrogel while stirring. Curcumin-based MN patches were fabricated similarly using the one-layer centrifugation method. CH-ARG/PVA/CUR hydrogel (5 mL) was poured into PDMS molds containing an array of 15×15 pyramids, each with a height of 345 μm and a base length of 250 μm. The molds were centrifuged at 1000 rpm for 10 minutes and then dried at 25° C. for 24 hours. After drying, the CUR-loaded microneedle patches were obtained which are hereafter referred to as CUR-MNs in the specification and drawing.

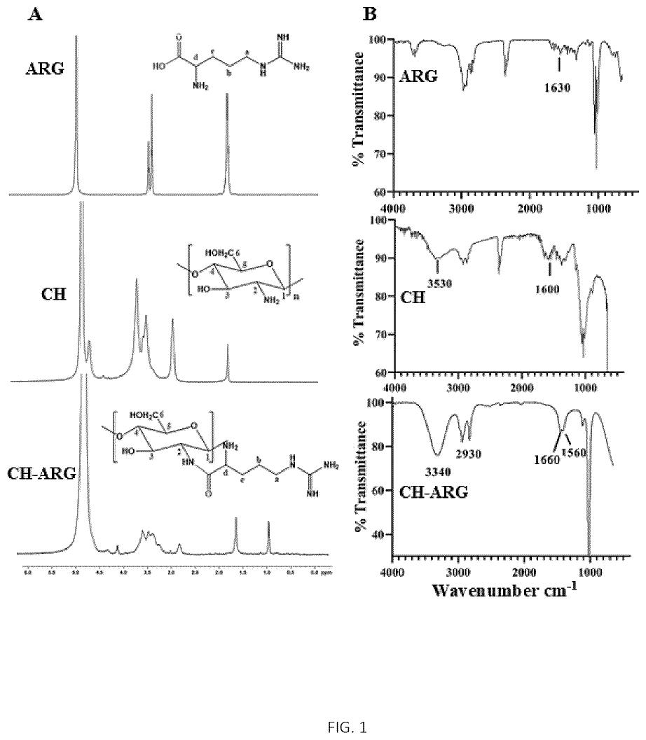

Compared to CH and ARG, the ¹H NMR spectrum of CH-ARG exhibited several notable changes, as shown in FIG. 1A. The protons of CH-ARG at positions a, b, and c were associated with three peaks at 0.95 ppm, 3.32 ppm, and 3.70 ppm, respectively. The additional peaks in the CH-ARG spectrum, which were absent in the spectra of pure CH and pure ARG, indicate the conjugation of chitosan and the amino acid arginine, resulting in the formation of CH-ARG.

FIG. 1B shows the fourier-transform infrared (FTIR) spectra of ARG, CH, and arginine-modified chitosan (CH-ARG). Compared to CH and ARG, the CH-ARG spectrum exhibited an additional band at 1560 cm⁻¹, presumably due to the formation of an amide bond between CH and ARG. The characteristic bands of the ARG guanidine group appeared at 1660 cm⁻¹. Additionally, the ratio of the methylene group was improved by the grafted ARG, resulting in a much stronger —CH₂— absorption peak at 2930 cm⁻¹ in the CH-ARG spectrum compared to that of simple chitosan. Furthermore, the peak at 3200-3580 cm⁻¹ in the CH-ARG spectrum became broader due to increased hydrogen-bond interactions, indicating successful grafting of ARG onto CH.

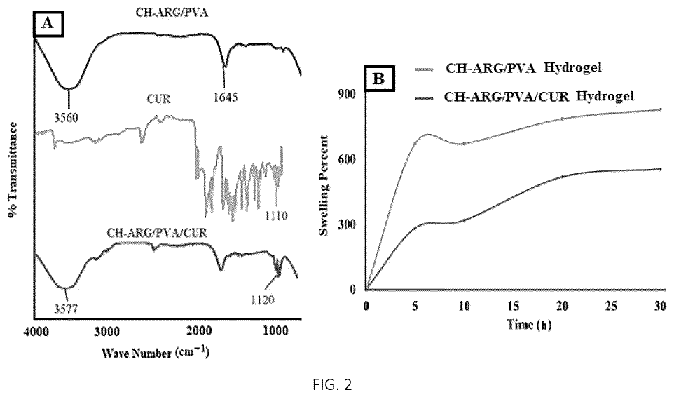

FIG. 2A shows the FTIR spectra of CH-ARG/PVA and CH-ARG/PVA/Curcumin hydrogels. By comparing the spectra of CH-ARG with the CH-ARG/PVA hydrogel, the absorption peak at 3340 cm⁻¹, characteristic of —OH and —NH stretching vibrations in CH-ARG, shifted to 3560 cm⁻¹ in the CH-ARG/PVA hydrogel due to -OH stretching vibrations present in PVA. The band at 1560 cm⁻¹ in the CH-ARG spectrum, corresponding to the Amide II region (specifically N—H bending vibration and symmetric-NH deformation), shifted to 1645 cm⁻¹ with increased intensity, along with the disappearance of the band at 1560 cm⁻¹. These findings suggest a secondary interaction between the two polymers.

FTIR analysis was also employed to study the interaction of curcumin (CUR) with the CH-ARG/PVA hydrogel. The comparative FTIR spectra of CH-ARG/PVA, CUR, and CH-ARG/PVA/Curcumin hydrogels are shown in FIG. 2A. The characteristic band of the ether linkage (C—O—C) of CUR was observed at 1120 cm⁻¹, indicating the presence of CUR in the CH-ARG/PVA/CUR hydrogel. Moreover, the stretching vibration at 3560 cm⁻¹, corresponding to OH/NH2 groups, shifted to 3577 cm⁻¹, revealing electrostatic interaction between CUR and both chitosan and PVA.

FIG. 2B shows the swelling behavior of the developed hydrogels. The potential of hydrogels to absorb a large quantity of water can be effective in eliminating wound exudates. The swelling at equilibrium in phosphate-buffered saline (PBS) of CH-ARG/PVA hydrogel (without the hydrophobic moiety, CUR) was 800±50%, while CH-ARG/PVA/CUR exhibited a maximum swelling of around 480±30%.

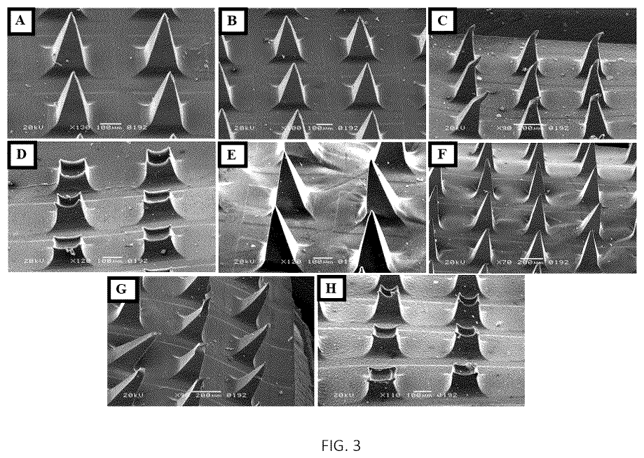

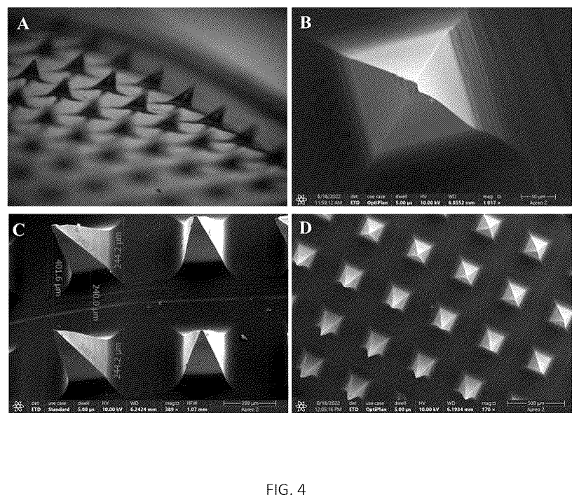

The MNs, after drying, were analyzed for their mechanical characteristics and surface morphology using scanning electron microscopy (SEM) (Model: JSM-5300, Japan). FIG. 3 shows the mechanical characteristics of blank-MNs and CUR-MNs. To overcome the resistive force of the skin, it is necessary for the load applied on the tip to exceed a resistance of approximately 3.18 MPa, which is the requisite pressure for penetrating human skin. Thus, a range of 100 to 1000 g was proposed to carry out the stress analysis, and the observed results are presented in FIG. 3. The results showed that the maximum stress at the tip of the MNs is 10.7 MPa, which is considerably higher than the 3.18 MPa pressure required to penetrate human skin. The mechanical strength of the MN tips used in this work is sufficient to easily penetrate the skin. The microscopic and SEM images of the prepared blank and CUR-MNs patches, illustrating the pyramid structure after demolding, are shown in FIG. 4. The height and base length of the individual pyramidal microneedles in the blank-MNs patches were found to be 340±5 μm and 244±3 μm, respectively, as displayed in the SEM images in FIG. 4. The height of the MNs was slightly lower than that of the PDMS mold, which could be attributed to the drying process at the end of molding. These heights are sufficient for these MNs to disrupt the stratum corneum of the skin.

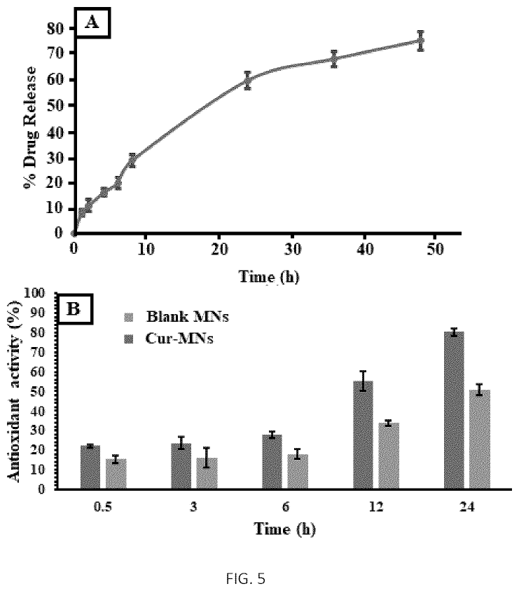

The in vitro release profile of CUR is presented in FIG. 5A. The CUR-MNs exhibited a slower release throughout the first 8 hours. Initially, only 15% of the drug was released from the MNs within 5 hours, which increased in a controlled manner, reaching 56% at 24 hours, indicating that the MNs maintained control over the entrapped drug. The cumulative release over a 48-hour period was 70%. As demonstrated in FIG. 5B, CUR-MNs exhibited comparatively higher antioxidant activity during the predetermined time interval, with a maximal scavenging capability of 80.40%. The results of the tests revealed that CUR retained its ability to scavenge free radicals even after encapsulation, as its reactive keto-enol component was protected from degradation when exposed to free radicals. As a result, CH and PVA were found to be effective in preserving CUR and retaining its antioxidant activity.

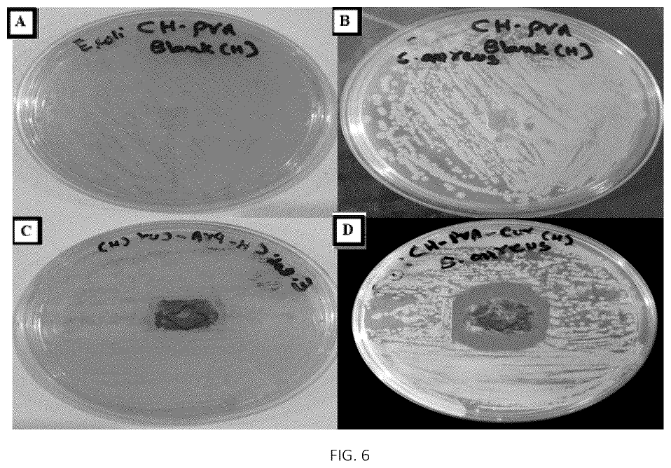

The zones of inhibition created by blank and CUR-MNs against both strains of Staphylococcus aureus and Escherichia coli are presented in FIG. 6(A-D). The zones of inhibition in CUR-MNs were found to be larger than those in blank-MNs. CUR-MNs exhibited notable antibacterial activity against both gram-positive and gram-negative bacteria. CUR encapsulated in MNs could effectively penetrate bacterial cell walls, leading to enhanced antibacterial action.

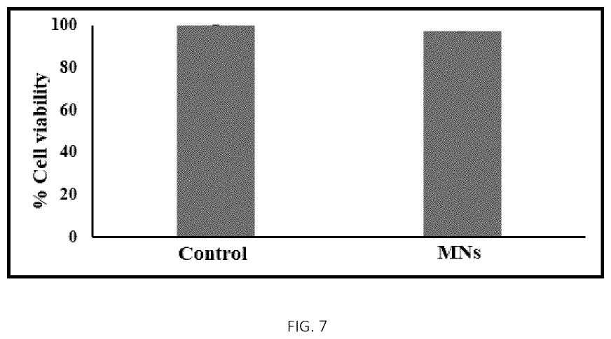

The in vitro cytotoxicity assays demonstrated that the developed MNs are not toxic to normal cells. As shown in FIG. 7, the cell viability of the MNs was above 97% after incubation for 24 h compared to the control which showed 100% cell survival. The good cell compatibility proved that the prepared MNs could be safe for further in vivo applications.

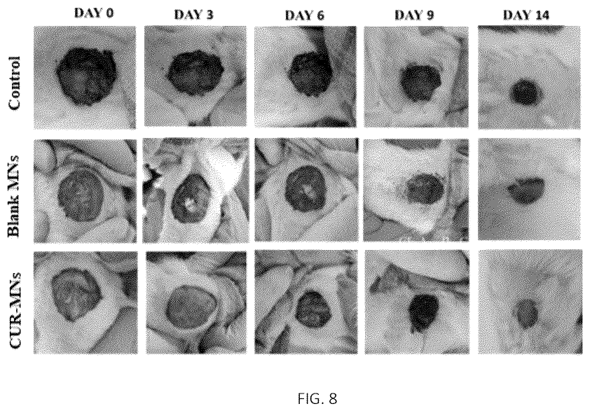

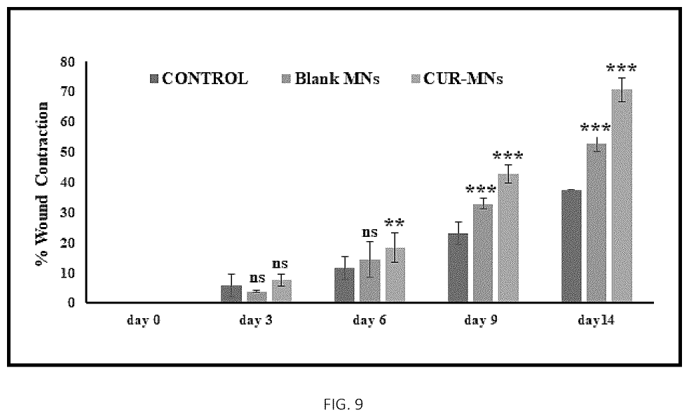

FIG. 8 shows the macroscopic visualization images of wound for blank-MNs, CUR-MNs, and control groups at 0, 3, 6, 9 and 14 day. The results indicated considerable wound healing on day 14 with no symptoms of infection, enhanced wound contraction, hair re-growth, skin architecture recovery, and tissue strength. Macroscopic investigation revealed no evidence of infection in either of the treated (blank-MNs and CUR-MNs) or control groups. However, earlier granulation tissue formation was observed in the treated groups than in the control groups at day 3, indicating faster healing progression in the treated wounds. In addition, the percentage of wound contraction, as illustrated in FIG. 9, was determined to assess wound closure. The CUR-MNs treatment group showed considerable wound healing from day 3 onwards, with 70±3.37% wound closure on day 14 compared to 52±1.67% and 37±2.86% wound closure in the blank-MNs and control groups, respectively.

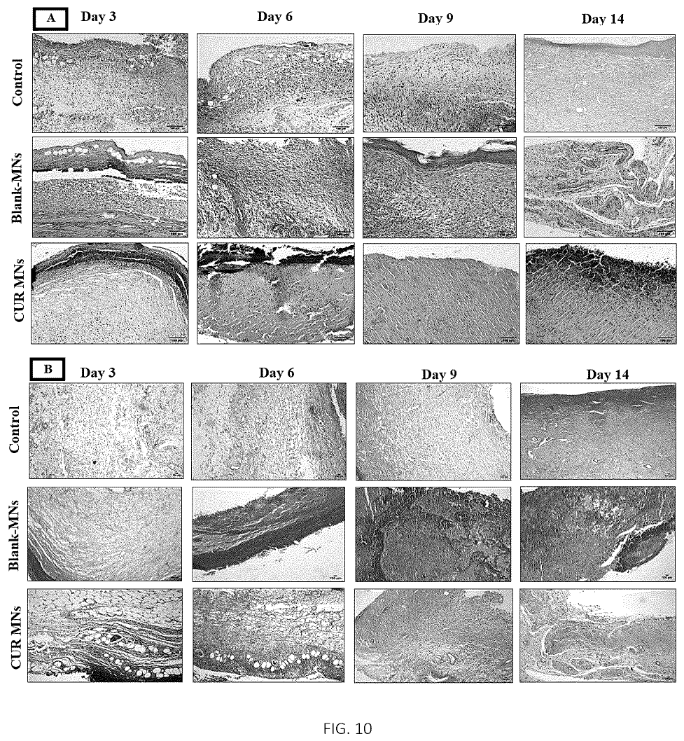

Histopathological analysis of both control and treatment groups (blank-MNs and CUR-MNs) showed complete tissue destruction at day 3 with increased inflammation as shown in FIG. 10. However, reduced inflammation was observed in the CUR-MNs treated group from day 3 onwards to day 6, with deposition of the collagen matrix indicated by the presence of green color. As shown in FIG. 10B. At day 14, intact dermis with collagen matrix formation was observed in the CUR-MNs group compared to the blank-MNs and control groups, which still showed mild infiltration of inflammatory cells with the disrupted dermis, as shown in FIGS. 10A and 10B. Hence, the developed CUR-MNs successively enhanced the wound healing potential by inducing collagen deposition, which was found to be essential in the formation of granulation tissue, promoting wound healing, and its rapid increase led to an efficient wound healing.

The microneedle patches may offer a promising approach for the transdermal delivery of bioactive compounds such as curcumin, enhancing their therapeutic effects in wound healing and other medical applications. These patches demonstrate strong mechanical properties, biocompatibility, and sustained release capabilities, making them suitable for diverse biomedical uses.

1: A hydrogel microneedle-based transdermal delivery patch, wherein the patch is fabricated using a hydrogel composition that comprises polyvinyl alcohol (PVA), arginine-modified chitosan, and a crosslinker, and can deliver one or more therapeutic agents transdermally.

2: The hydrogel microneedle-based transdermal delivery patch of claim 1, wherein the arginine-modified chitosan is prepared by modifying 5 mL of a 0.3% (w/v) chitosan solution at pH 5.5 with 3 mL of a 0.04 mM arginine solution, preactivated with N-hydroxysuccinimide and 1-ethyl-3-(3-dimethylaminopropyl)carbodiimide.

3: The hydrogel microneedle-based transdermal delivery patch of claim 1, wherein the concentration of PVA ranges from 10% to 12% (w/v).

4: The hydrogel microneedle-based transdermal delivery patch of claim 1, wherein the arginine-modified chitosan and PVA solutions are mixed in a 1:2 ratio by volume.

5: The hydrogel microneedle-based transdermal delivery patch of claim 1, wherein the concentration of the crosslinker, trimethyl orthoformate (TMOF), is 4% (v/v) relative to the total volume of the polymeric mixture.

6: The hydrogel microneedle-based transdermal delivery patch of claim 1, wherein the patch comprises a 15×15 array of pyramidal microneedles, with each microneedle having an average height of 340±5 μm and an average base length of 244±3 μm.

7: The hydrogel microneedle-based transdermal delivery patch of claim 1, wherein the patch is designed for the transdermal delivery of curcumin as a therapeutic agent, and further incorporates 1 mL of 2 mg/mL methanolic curcumin.

8: The hydrogel microneedle-based transdermal delivery patch of claim 7, wherein the curcumin amount is 120 μg per patch.

9: The hydrogel microneedle-based transdermal delivery patch of claim 7, wherein the patch demonstrates a 70% release of the loaded curcumin over a 48-hour period in a controlled-release manner.

10: The hydrogel microneedle-based transdermal delivery patch of claim 7, wherein the patch demonstrates healing of animal wounds.
